(19) United States
(12) Patent Application Publication
(10) Pub. No.: US 2005/0256100 A1
(43) Pub. Date: Nov. 17, 2005

(54) Protease inhibitors

(76) Inventors: Jae U. Jeong, Collegeville, PA (US); Dennis . Yamashita, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/514,965

(22) PCT Filed: May 21, 2003
(86) PCT No.: PCT/US03/16254

§ 371 Date: Nov. 18, 2004

Related U.S. Application Data
(60) Provisional application No. 60/382,514, filed on May 22, 2002.

(51) Int. Cl. A61K 31/55; C07D 413/02; C07D 43/02

(52) U.S. Cl. 514/212.03; 514/212.08; 540/524; 540/526

(57) ABSTRACT

This invention relates in general to certain substituted 3,7-dioxoazepan-4-ylamides of formula 1 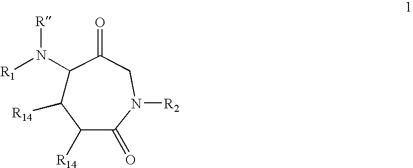 which are protease inhibitors.

### BACKGROUND OF INVENTION

This invention relates in general to certain substituted 3,7-dioxoazepan-4-ylamides which are protease inhibitors. More particularly they are inhibitors of cysteine and serine proteases, particularly compounds which inhibit cysteine proteases. More specifically these compounds inhibit cysteine proteases of the papain superfamily, including, in particular those of the cathepsin family, most particularly cathepsin K Such compounds are useful for treating diseases in which cysteine proteases are implicated, especially diseases of excessive bone or cartilage loss, e.g., osteoporosis, periodontitis, and arthritis; and certain parasitic diseases, e.g., malaria.

Cathepsins are a family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J. Biol. Chem. 271, 12517-12524; Drake, F. H., et al., (1996) J. Biol. Chem. 271, 12511-12516; Bromme, D., et al., (1996) J. Biol. Chem. 271, 2126-2132.

Cathepsin K has also been variously denoted as cathepsin O or cathepsin O2 in the literature. The designation cathepsin K is considered to be the most appropriate one.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and the like.—See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2,445-458.

Cathepsin K is believed to play a causative role in diseases of excessive bone or cartilage loss. Bone is composed of a protein matrix in which spindle- or plate-shaped crystals of hydroxyapatite are incorporated. Type I collagen represents the major structural protein of bone comprising approximately 90% of the protein matrix. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodelling at discrete foci throughout life. These foci, or remodelling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

Several published studies have demonstrated that inhibitors of cysteine proteases are effective at inhibiting osteoclast-mediated bone resorption, and indicate an essential role for cysteine proteases in bone resorption. For example, Delaisse, et al., Biochem. J., 1980,—192, 365, disclose a series of protease inhibitors in a mouse bone organ culture system and suggest that inhibitors of cysteine proteases (e.g., leupeptin, Z-Phe-Ala-CHN2) prevent bone resorption, while serine protease inhibitors were ineffective. Delaisse, et al., Biochem. Biophys. Res. Commun., 1984, 125, 441, disclose that E-64 and leupeptin are also effective at preventing bone resorption in vivo, as measured by acute changes in serum calcium in rats on calcium deficient diets. Lerner, et al., J. Bone Min. Res., 1992, 7, 433, disclose that cystatin, an endogenous cysteine protease inhibitor, inhibits PIH stimulated bone resorption in mouse calvariae. Other studies, such as by Delaisse, et al., Bone, 1987,—8, 305, Hill, et al., J. Cell. Biochem., 1994, 56, 118, and Everts, et al., J. Cell. Physiol.,—1992, 150, 221, also report a correlation between inhibition of cysteine protease activity and bone resorption. Tezuka, et al., J. Biol. Chem., 1994, 269, 1106, Inaoka, et al., Biochem. Biophys. Res. Commun., 1995, 206, 89 and Shi, et al., FEBS Lett., 1995, 357, 129 disclose that under normal conditions cathepsin K, a cysteine protease, is abundantly expressed in osteoclasts and may be the major cysteine protease present in these cells.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

We have now discovered a novel class of substituted substituted 3,7-dioxoazepan-4-ylamides which are protease inhibitors, most particularly of cathepsin K.

### SUMMARY OF INVENTION

The present invention provides protease inhibitors of formula I which inhibit the likes of cathepsin K, and which are useful for treating diseases which may be therapeutically modified by altering the activity of such proteases.

Accordingly, in the first aspect, this invention provides a compound according to Formula I. 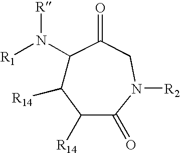
wherein:
R₁ is either formula A or B 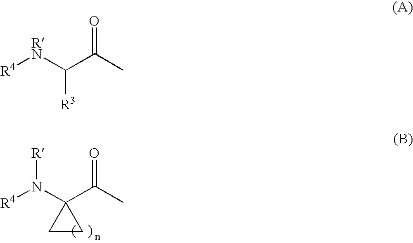
   wherein in formula (B), n is an integer from 1 to 5;
R₂ is H, C₁₋₆alkyl, C₃₋₆cycloalkyl-C₀₋₆alkyl, Ar—C₀₋₆alkyl, Het-C₀₋₆alkyl, R₉C(O)—, R₉C(S)—, R₉SO₂—, R₉OC(O)—,
R₉R₁₁NC(O)—, R₉R₁₁NC(S)—, R₉(R₁₁)NSO₂— 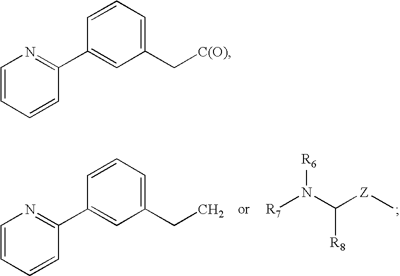
R₃ is H, C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, Ar—ArC₀₋₆alkyl, Ar-HetC₀₋₆alkyl, Het-ArC₀₋₆alkyl, or Het-HetC₀₋₆alkyl;
R₃ and R′ may be connected to form a pyrrolidine, piperidine or morpholine ring;
R₄ is C₁₋₆alkyl, C₃₋₆cycloalkyl-C₀₋₆alkyl, Ar—C₀₋₆alkyl, Het-C₀₋₆alkyl, R₅C(O), R₅—C(S)—, R₅SO₂—, R₅OC(O)—, R₅R₁₂NC(O)—, or R₅R₁₂NC(S)—;
R₅ is H, C₁₋₆alkyl, C₂₋₆alkenyl, C₂₋₆alkynyl, C₃₋₆cycloalkyl-C₀₋₆alkyl, C₂₋₆-alkanonyl, Ar—C₀₋₆alkyl, Het-C₀₋₆alky Ar—ArC₀₋₆alkyl, Ar-HetC₀₋₆alkyl, Het-ArC₀₋₆alkyl, or Het-HetC₀₋₆alkyl;
R₆ is H, C₁₋₆alkyl, Ar—C₀₋₆alkyl, or Het-C₀₋₆alkyl;
R₇ is H, C₁₋₆alkyl, C₃₋₆cycloalkyl-C₀₋₆alkyl, Ar—C₀₋₆alkyl, Het-C₀₋₆alkyl, R₁₀C(O)—, R₁₀C(S)—, R₁₀SO₂—, R₁₀OC(O)—, R₁₀R₁₃NC(O)—, or R₁₀R₁₃NC(S)—;
R₈ is H, C₁₋₆alkyl, C₂₋₆alkenyl, C₂₋₆alkynyl, Ar—C₀₋₆alkyl or Het-C₀₋₆alkyl;
R₉ is C₁₋₆alkyl, C₃₋₆cycloalkyl-C₀₋₆alkyl, Ar—C₀₋₆alkyl or Het-C₀₋₆alkyl;
R₁₀ is C₁₋₆alkyl, C₃₋₆cycloalkyl-C₀₋₆alkyl, Ar—C₀₋₆alkyl or Het-C₀₋₆alkyl;
R₁₁ is H, C₁₋₆alkyl, Ar—C₀₋₆alkyl, C₃₋₆cycloalkyl-C₀₋₆alkyl, or Het-C₀₋₆alkyl;
R₁₂ is H, C₁₋₆alkyl, Ar—C₀₋₆alkyl, or Het-C₀₋₆alkyl;
R₁₃ is H, C₁₋₆alkyl, Ar—C₀₋₆alkyl, or Het-C₀₋₆alkyl;
each R₁₄ is independently H, C₁₋₆alkyl, OC₁₋₄alkyl, SC₁₋₄alkyl, N(R₁₂)₂, —CH₂OC₁₋₄alkyl, CH₂SC₁₋₄alkyl, CH₂N(R₁₂)₂, Ar—C₀₋₆alkyl or Het-C₀₋₆alkyl;
R′ is H, C₁₋₆alkyl, Ar—C₀₋₆alkyl, or Het-C₀₋₆alkyl;
R″ is H, C₁₋₆alkyl, Ar—C₀₋₆alkyl, or Het-C₀₋₆alkyl;
Z is C(O) or CH₂; or
a pharmaceutically acceptable salt, hydrate or solvate thereof.

In another aspect, this invention provides a pharmaceutical composition comprising a compound according to Formula I and a pharmaceutically acceptable carrier, diluent or excipient.

In yet another aspect, this invention provides intermediates useful in the preparation of the compounds of Formula I.

In still another aspect, this invention provides a method of treating diseases in which the disease pathology may be therapeutically modified by inhibiting proteases, particularly cysteine and serine proteases, more particularly cysteine proteases, even more particularly cysteine proteases of the papain superfamily, yet more particularly cysteine proteases of the cathepsin family, most particularly cathepsin K.

In a particular aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis; and for treating certain parasitic diseases, such as malaria.

### DETAILED DESCRIPTION

Definitions and Preferred Embodiments

The present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

Hydrogen” or “H” includes all of its possible isotopes, including deuterium and tritium.

C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. C₁₋₆alkyl may be optionally substituted by a moiety selected from the group consisting of: OR₁₅, C(O)R₁₅, SR₁₅, S(O)R—₁₅, S(O)₂R₁₅, N(R₁₅)₂ R₁₅NC(O)OR₁₆, CO₂R₁₅, CO₂N(R₁₅)₂N(C═NH)NH₂, Het, C₃₋₆-cycloalkyl, and Ar; where R₁₅ is: H, C₁₋₆alkyl, Ar—C₀₋₆alkyl, or Het-C₀₋₆alkyl; and R₁₆ is: H, C₁₋₆alkyl, C₂₋₆alkenyl, C₂₋₆alkynyl, C₃₋₆cycloalkyl-C₀₋₆alkyl, Ar—C₀₋₆alkyl or Het-C₀₋₆alkyl.

C₃₋₆cycloalkyl” as applied herein is meant to include substituted and unsubstituted cyclopropane, cyclobutane, cyclopentane and cyclohexane.

C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

C₂₋₆alkanonyl” as applied herein is meant to include unsubstituted and substituted acetyl, propanonyl, butanonyl, pentanonyl, and hexanonyl

C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

Halogen” means F, Cl, Br, and I.

As used herein “Het” or “heterocyclic” represents a stable 5- to 7-membered monocyclic, stable a 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆alkyAr, C₁₋₆alkyl, OR₁₇, N(R₁₇)₂, SR₁₇, S(O)R₁₅, S(O)₂R₁₅, CF₃, NO₂, CN, CO₂R₁₇, CON(R₁₇), F, Cl, Br and I, where R₁₇ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, 1-oxo-pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, quinoxalinyl, isoquinolinyl, benzimidazolyl, benzopyranyl, benzoxazolyl, furanyl, benzofuranyl, thiophenyl, benzo[b]thiophenyl, thieno[3,2-b]thiophenyl, benzo[1,3]dioxolyl, 1,8-naphthyridinyl, pyranyl, tetrahydrofuranyl, tetrahydropyranyl, thienyl, benzoxazolyl, thiamorpholinyl sulfoxide, thiamorpholinyl sulfone, and oxadiazolyl, as well as triazolyl, thiadiazolyl, oxadiazolyl, isothiazolyl, imidazolyl, pyridazinyl, pyrimidinyl, triazinyl and tetrazinyl which are available by routine chemical synthesis and are stable. The term heteroatom as applied herein refers to oxygen, nitrogen and sulfur.

“Ar” or “aryl” means phenyl or naphthyl, optionally substituted by one or more of Ph-C₀₋₆alkyl; Het-C₀₋₆alkyl; C₁₋₆alkoxy; Ph-C₀₋₆alkoxy; Het-C₀₋₆alkoxy; OH, (CH₂)₁₋₆NR₁₅R—₁₆; O(CH₂)₁₋₆NR₁₅R₁₆; C₁₋₆alkyl, OR₁₇, N(R₁₇)₂, SR₁₇, S(O)R₁₅, S(O)₂R₁₅, CF₃, NO₂, CN, CO₂R₁₇, CON(R₁₇), F, Cl, Br or I; where R₁₅ and R₁₆ are H, C₁₋₆alkyl, Ph-C₀₋₆alkyl, naphthyl-C₀₋₆alkyl or Het-C₀₋₆alkyl; and R₁₇ is phenyl, naphthyl, or C₁₋₆alkyl.

“Ar—Ar” means aryl covalently linked to a second aryl. Examples of “Ar—Ar” include biphenyl or naphythyl-pheny or phenyl-naphthyl.

“Ar—Het” means an aryl group covalently linked to a heterocycle. Examples of “Ar—Het” include phenyl-piperidine, phenyl-piperazine, phenyl-2-oxopiperazine, naphthyl-piperidine, naphthyl-piperazine, and naphhyl-2-oxopiperazine.

“Het-Ar” means a heterocycle covalently linked to a aryl group. Examples of such “Het-Ar” include piperidinyl-phenyl, piperazinyl-phenyl, 2-oxopiperazinyl-phenyl, piperidinyl-naphthyl, piperazinyl-naphthyl, and 2-oxpiperazinyl-naphthyl.

“Het-Het” means a heterocycle covalently linked to a second heterocycle. Examples of such “Het-Het” include bipyridine, pyridinyl-piperidine, pyridinyl-piperazine, pyridinyl-2-oxopiperazine, thiophenyl-piperidine, thiophenyl-piperazine, and thiophnyl-2-oxopiperazine.

Here and throughout this application the term C₀ denotes the absence of the substituent group immediately following. For instance, in the moiety ArC₀₋₆alkyl, when C is 0 (zero), the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein: t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein: m-CPBA refers to 3-chloroperoxybenzoic acid, EDC refers to N-ethyl-N′-(dimethylaminopropyl)-carbodiimide, DMF refers to dimethyl formamide, DMSO refers to dimethyl sulfoxide, TEA refers to triethylamine, TFA refers to trifluoroacetic acid, and THF refers to tetrahydrofuran.

The following definitions set out preferred embodiments of this invention as regards Formula 1.

### Preferred Embodiments

In compounds of Formula I, when R₁ is 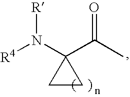
n is preferably 4, to provide 1-amino-1-acyl cyclohexane compounds. The cycloalkyl ring may be unsubstituted or substituted with one or more of C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, or halogen.

The cycloalkyl ring is more preferably unsubstituted.

In compounds of Formula I, when R₁ is 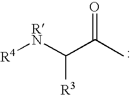
R₃ is H, C₁₋₆-allyl, C₃₋₆cycloalkyl-C₀₋₆alkyl, C₂₋₆alkenyl, C₂₋₆alkynyl, Het-C₀₋₆alkyl, Ar—C₀₋₆alkyl, Ar—ArC₀₋₆alkyl, Ar-HetC₀₋₆alkyl, Het-ArC₀₋₆alkyl, or Het-HetC₀₋₆alkyl.
R₃ is preferably H, C₃₋₆cycloalkyl-C₀₋₆alkyl, Ar—C₀₋₆alkyl, or C₁₋₆alkyl.
R₃ is more preferably H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluyl, naphthalen-2-ylmethyl, benzyloxymethyl, or hydroxymethyl.
R₃ is even more preferably toluyl, isobutyl or cyclohexylmethyl.
R₃ is most preferably isobutyl.
R₄ is H, C₁₋₆alkyl, C₃₋₆cycloalkyl-C₀₋₆alkyl, Ar—C₀₋₆alkyl, Het-C₀₋₆alkyl, R₅C(O)—, R₅C(S)—, R₅SO₂—, R₅OC(O)—, R₅R₁₂NC(O)—, or R₅R₁₂NC(S)—.
R₄ is more preferably R₅OC(O)—, R₅C(O)— or R₅SO₂—.
R₄ is most preferably R₅C(O)—.

In some embodiments, R₄ is preferably methanesulfonyl.

Preferably R₅ is C₁₋₆alkyl, C₂₋₆alkenyl, C₃₋₆cycloalkyl-C₀₋₆alkyl, C₂₋₆alkanonyl, Ar—C₀₋₆alkyl or Het-C₀₋₆alkyl.

More preferably, and especially when R₄ is R₅C(O)—, where R₅ is methyl, especially halogenated methyl, more especially trifluoromethyl, especially C₁₋₆alkoxy and aryloxy substituted methyl, more especially phenoxy-methyl, 4-fluoro-phenoxy-methyl, especially heterocycle substituted methyl, more especially 2-thiophenyl-methyl;
butyl, especially aryl substituted butyl, more especially 4-(4-methoxy)phenyl-butyl;
isopentyl;
cyclohexyl;
pentanonyl, especially 4-pentanonyl;
butenyl, especially aryl substituted butenyl, more especially 4,4-bis(4 methoxyphenyl)but-3-enyl;
phenyl, especially phenyl substituted with one or more halogens, more especially 3,4-dichlorophenyl and 4-fluorophenyl, especially phenyl substituted with one or more C₁₋₆alkoxy or aryloxy groups, more especially 3,4-dimethoxy-phenyl, 3-benzyloxy-4-methoxy-phenyl, especially phenyl substituted with one or more sulfonyl groups, more especially 4-methanesulfonyl-phenyl;
benzyl;
naphthalenyl, especially naphthylen-2-yl;
benzo[1,3]dioxolyl, especially benzo[1,3]dioxol-5-yl, furanyl, especially furan-2-yl, especially substituted furanyl, such as 5-nitro-furan-2-yl, 5-(4-nitrophenyl)-furan-2-yl, 5-(3-trifluoromethyl-phenyl)-furan-2-yl, more especially halogen substituted furanyl, even more especially 5-bromo-furan-2-yl, more especially aryl substituted furanyl, even more especially 5-(4-chloro-phenyl)-furan-2-yl;
tetrahydrofuranyl, especially tetrahydrofuran-2-yl;
benzofuranyl, especially benzofuran-2-yl, and especially C₁₋₆alkoxy substituted benzofuranyl, more especially 5-(2-piperazin-4-carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; 7-methoxybenzofuran-2-yl, 5-methoxy-benzofura-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C₁₋₆alkyl substituted benzofuranyl, most especially 3-methyl-benzofuran-2-yl;
benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl;
quinolinyl, especially quinolin-2-yl, quinolin-3-yl, quinolin-4-yl, quinolin-6-yl, or quinolin-8-yl;
quinoxalinyl, especially quinoxalin-2-yl;
1,8-naphthyridinyl, especially 1,8-naphthyridin-2-yl;
indolyl, especially indol-2-yl, especially indol-6-yl, indol-5-yl, especially C₁₋₆alkyl substituted indolyl, more especially N-methyl-indol-2-yl;
pyridinyl, especially pyridin-2-yl , pyridin-5-yl, especially 1-oxy-pyridin-2-yl, especially C₁₋₆alkyl substituted pyridinyl, more especially 2-methyl-pyridin-5-yl;
furo[3,2-b]pyridinyl, especially furo[3,2-b]pyridin-2-yl, and C₁₋₆alkyl substituted furo[3,2-b]pyridinyl, especially 3-methyl-furo[3,2-b]pyridin-2-yl;
thiophenyl, especially thiophen-3-yl, especially C₁₋₆alkyl substituted thiophenyl, more especially 5-methyl-thiophen-2-yl, especially halogen substituted thiophenyl, more especially 4,5-dibromo-thiophen-2-yl;
thieno[3,2-b]thiophene, especially thieno[3,2-b]thiophene-2-yl, more especially C₁₋₆alkyl substituted thieno[3,2-b]thiophene-2-yl, more especially 5-tert-butyl-3-methyl-thieno[3,2-b]thiophene-2-yl;
isoxazolyl, especially isoxazol-4-yl, especially C₁₋₆-alkyl substituted isoxazolyl, more especially 3,5-dimethyl-isoxazol-4-yl;
oxazolyl, especially oxazol-4-yl, more especially 5-methyl-2-phenyl oxazolyl, or 2-phenyl-5-trifluoromethyl-oxazol-4-yl.

When R₄ is R₅SO₂, R₅ is preferably pyridin-2-yl or 1-oxo-pyridin-2-yl.
R′ is preferably H or naphthalen-2-yl-methyl. Most preferably R′ is H.
R″ is most preferably H or C₁₋₆-alkyl.
R₁₄ is most preferably H, C₁₋₆alkyl, especially is methyl, ethyl, propyl, butyl, pentyl or hexyl, more especially methyl.

Preferably R₂ is C₁₋₆ alkyl, C₃₋₆cycloalkyl-C₀₋₆-alkyl, Ar—C₀₋₆alkyl, R₉C(O)—, R₉SO₂,
R₉R₁₁NC(O)—, or 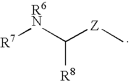
More preferably R₂ is C₁₋₆alkyl, C₃₋₆cycloalkyl-C₀₋₆-alkyl, Ar—C₀₋₆alkyl. Most preferably R₂ is R₉SO₂ or C₃₋₆cycloalkyl-C₀₋₆-alkyl.

In such embodiments R₆ is preferably H, C₁₋₆alkyl, Ar—C₀₋₆alkyl, or Het-C₀₋₆alkyl; more preferably H.

In addition, in such embodiments, R₇ is is preferably R₉OC(O); R₈ is preferably C₁₋₆alkyl, more preferably isobutyl; and R₉ is C₁₋₆alkyl, C₃₋₆cycloalkyl-C₀₋₆alkyl, Ar—C₀₋₆alkyl, and Het-C₀₋₆alkyl.

More preferably, in such embodiments, R₉ is methyl; ethyl, especially C₁₋₆alkyl-substituted ethyl, more especially 2-cyclohexyl-ethyl; butyl, especially C₁₋₆butyl, more especially 3-methylbutyl; tert-butyl, particularly when R₂ is R₉OC(O); isopentyl; phenyl, especially halogen substituted phenyl, more especially 3,4-dichlorophenyl, 4-bromophenyl, 2-fluorophenyl, 4-fluorophenyl, 3-chlorophenyl, 4-chlorophenyl, especially C₁₋₆-alkoxy phenyl, more especially 3-methoxyphenyl, 4-methoxyphenyl, 3,4-dimethoxyphenyl, especially cyanophenyl, more especially 2-cyanophenyl; toluyl, especially Het-substituted toluyl, more especially 3-(pyridin-2-yl)toluyl; naphthylenyl, especially naphthylen-2-yl; benzoyl, especially 2-benzoyl; benzo[1,3]dioxolyl, especially benzo[1,3]dioxol-5-yl; benzo[1,2,5]oxadiazolyl, especially benzo[1,2,5]oxadiazol-4-yl; pyridinyl, especially pyridin-2-yl, pyridin-3-yl, especially 1-oxy-pyridinyl, more especially 1-oxy-pyridin-2-yl, 1-oxy-pyridin-3-yl; especially C₁₋₄alkylpyridinyl, more especially 3-methyl-pyridin-2-yl, 6 methyl-pyridin-2-yl, thiophene, especially thiophene-2-yl; thiazolyl, especially thiazol-2-yl; 1H-imidazolyl, especially 1H-imidazol-2-yl, 1H-imidazol-4-yl, more especially C₁₋₆alkyl substituted imidazolyl, even more especially 1-methyl-1H-imidazol-2-yl, 1-methyl-1H-imidazol-4-yl; 1H-[1,2,4]triazolyl, especially 1H-[1,2,4]triazol-3-yl, more especially C₁₋₆alkyl substituted 1H-[1,2,4]triazolyl, even more especially 5-methyl-1H-[1,2,4]triazol-3-yl; or quinolinyl.

When R₂ is R₉SO₂, R₉ is most preferably pyridin-2-yl or 1-oxy-pyridin-2-yl; and R₁₀ is preferably C₁₋₆alkyl, Ar—C₀₋₆alkyl or Het-C₀₋₆alkyl.
Z is preferably C(O) or CH₂.
R₂ is also preferably H, cyclohexyl, methylcyclohexyl, toluyl, aryl substituted ethyl, especially 2-phenyl ethyl, or 2-[3-(pyridin-2-yl) phenyl] ethyl.

Compounds of Formula I where R″ is H are preferred.

More preferred are compounds of Formula I wherein:
R₁ is 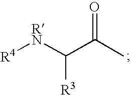
   where:
   R₂ is as defined above;
   R₃ is H, C₁₋₆alkyl, C₃₋₆cycloalkyl-C₀₋₆alkyl, or Ar—C₀₋₆alkyl;
   R₄ is R₅C(O)—, R₁₄SO₂—, or R₅OC(O)—;
   R₅ is C₁₋₄alkyl, C₂₋₄alkenyl, C₃₋₄cycloalkyl-C₀₋₆alkyl, C₄alkanonyl, Ar—C₀₋₆alkyl or Het-C₀₋₆alkyl;
   R₆ is H, C₁₋₆alkyl, Ar—C₀₋₆alkyl, or Het-C₀₋₆alkyl;
   R₇ is H, C₁₋₆alkyl, C₃₋₆cycloalky-C₀₋₆alkyl, Ar—C₀₋₆alkyl, Het-C₀₋₆alkyl, R₁₀C(O)—,
   R₈ is H, C₁₋₆alkyl, C₂₋₆alkenyl, C₂₋₆alkynyl, Hets₆alkyl or ArC₀₋₆alky;
   R₉ is C₁₋₆alkyl, C₃₋₆cycloalkyl-C₀₋₆alkyl, Ar—C₀₋₆alkyl or Het-C₀₋₆alkyl;
   R₁₀ is C₁₋₆alkyl, C₃₋₆cycloalkyl-C₀₋₆alkyl, Ar—C₀₋₆alkyl or Het-C₀₋₆alkyl;
   R₁₁ is H, C₁₋₆alkyl, Ar—C₀₋₆alkyl, C₃₋₆cycloalkyl-C₀₋₆alkyl, or Het-C₀₋₆alkyl;
   R₁₂ is H, C₁₋₆allyl, Ar—C₀₋₆alkyl, or Het-C₀₋₆alkyl;
   R₁₃ is H, C₁₋₆alkyl, Ar—C₀₋₆alkyl, or Het-C₀₋₆alkyl;
   each R₁₄ is independently H, C₁₋₆alkyl, C₂₋₆alkenyl, C₃₋₆cycloalkyl-C₀₋₆-alkyl, C₂₋₆alkanonyl, Ar—C₀₋₆alkyl or Het-C₀₋₆alkyl;
   R′ is H; and
   R″ is H; and
   Particularly preferred are such compounds wherein R₃ is isobutyl.

Still more preferred are compounds of Formula I wherein:
R₁ is 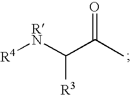
   R₂ is H, C₃₋₆cycloakyl-C₀₋₆alkyl, Ar—C₀₋₆alkyl, R₉C(O)—, R₉SO₂, R₉R₁₁NC(O)—, or 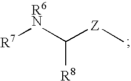
   R₃ is H, C₁₋₆alkyl, C₃₋₆cycloalkyl-C₀₋₆alkyl, or Ar—C₀₋₆alkyl;
   R₄ is R₅OC(O)—, R₅C(O)— and R₅SO₂—;
   R₅ is C₁₋₄alkyl, C₂₋₆alkenyl, C₃₋₆cycloalkyl-C₀₋₆alkyl, C₂₋₆alkanonyl, Ar—C₀₋₆alkyl or Het-C₀₋₆alkyl;
   R₆ is H;
   R₇ is R₁₀OC(O);
   R₈ is C₁₋₆alkyl;
   R₉ is C₁₋₆alkyl, C₃₋₆cycloalkyl-C₀₋₆alkyl, Ar—C₀₋₆alkyl or Het-C₀₋₆alkyl;
   R₁₀ is C₁₋₆alkyl, Ar—C₀₋₆alkyl or Het-C₀₋₆alkyl;
   R₁₁ is C₁₋₆alkyl, C₃₋₆cycloalkyl-C₀₋₆alkyl, Ar—C₀₋₆alkyl or Het-C₀₋₆alkyl;
   R′ is H; and
   R″ is H;
   Even more preferred are such compounds of Formula I wherein R₂ is Ar—C₀₋₆alkyl, R₉C(O)—, or R₉SO₂.

Yet more preferred are compounds of Formula I wherein:
R₁ is 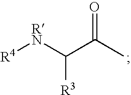
R₂ is C₃₋₆cycloalkyl-C₀₋₆alkyl or Ar—C₀₋₆alkyl;
R₃ is H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluyl, naphthalen-2-ylmethyl, benzyloxymethyl, or hydroxymethyl;
R₄ is R₅C(O)—;
R₅ is hydrogen, methyl, especially halogenated methyl, more especially trifluoromethyl, especially C₁₋₆-alkoxy and aryloxy substituted methyl, more especially phenoxy-methyl, 4-fluoro-phenoxy-methyl, especially heterocycle substituted methyl, more especially 2-thiophenyl-methyl; butyl, especially aryl substituted butyl, more especially 4 (4-methoxy)phenyl-butyl; isopentyl; cyclohexyl; pentanonyl, especially 4-pentanonyl; butenyl, especially aryl substituted butenyl, more especially 4,4-bis(4-methoxyphenyl)-but-3-enyl; phenyl, especially phenyl substituted with one or more halogens, more especially 3,4-dichlorophenyl and 4-fluorophenyl, especially phenyl substituted with one or more C₁₋₆alkoxy or aryloxy groups, more especially 3,4-dimethoxy-phenyl, 3-benzyloxy-4-methoxy-phenyl, especially phenyl substituted with one or more sulfonyl groups, more especially 4-methanesulfonyl-phenyl; benzyl; naphthylen-2-yl; benzo[1,3]dioxolyl, especially benzo[1,3]dioxol-5-yl, furanyl, especially furan-2-yl, especially substituted furanyl, such as 5-nitro-furan-2-yl, 5-(4-nitrophenyl)-furan-2-yl, 5-(3-trifluoromethyl-phenyl)-furan-2-yl, more especially halogen substituted furanyl, even more especially 5-bromo-furan-2-yl, more especially aryl substituted furanyl, even more especially 5-(4-chloro-phenyl)-furan-2-yl; tetrahydrofuran-2-yl; benzofuranyl, especially benzofuran-2-yl, and especially C₁₋₆-alkoxy substituted benzofuranyl, more especially 5-(2-piperazin carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl, 7-methoxy-benzofuran-2-yl, 5-methoxy-benzofuran-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C₁₋₆alkyl substituted benzofuranyl, most especially 3-methyl-benzofuran-2-yl; benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl; quinolinyl, especially quinolin-2-yl, quinolin-3-yl, quinolin-4-yl, quinolin-6-yl, and quinolin-8-yl; quinoxalinyl, especially quinoxalin-2-yl; 1,8-naphthyridinyl, especially 1,8-naphthyridin-2-yl; indolyl, especially indol-2-yl, especially indol-6-yl, indol-5-yl, especially C₁₋₆alkyl substituted indolyl, more especially N-methyl-indol-2-yl; pyridinyl, especially pyridin-2-yl , pyridin-5-yl, especially 1-oxy-pyridin-2-yl, especially C₁₋₆alkyl substituted pyridinyl, more especially 2-methyl-pyridin-5-yl; furo[3,2-b]pyridinyl, especially furo[3,2-b]pyridin-2-yl, and C₁₋₆alkyl substituted furo[3,2-b]pyridinyl, especially 3-methyl-furo[3,2-b]pyridin-2-yl; thiophenyl, especially thiophen-3-yl, especially C₁₋₆alkyl substituted thiophenyl, more especially 5-methyl-thiophen-2-yl, especially halogen substituted thiophenyl, more especially 4,5-dibromo-thiophen-2-yl; thieno[3,2-b]thiophene, especially thieno[3,2-b]thiophene-2-yl, more especially C₁-alkyl substituted thieno[3,2-b]thiophene-2-yl, more especially 5-tert-butyl-3-methyl-thieno[3,2-b]thiophene-2-yl; isoxazolyl, especially isoxazol-4-yl, especially C₁₋₆alkyl substituted isoxazolyl, more especially 3,5-dimethyl-isoxazol-4-yl; or oxazolyl, especially oxazol-4-yl, more especially 5-methyl-2-phenyl oxazol-yl, 2-phenyl-5-trifluoromethyl-oxazolyl;
R₉ is methyl; ethyl, especially C₁₋₆alkyl-substituted ethyl, more especially 2-cyclohexyl-ethyl; butyl, especially C₁₋₆butyl, more especially 3-methylbutyl; tert-butyl, particularly when R₂ is R₉OC(O); isopentyl; phenyl, especially halogen substituted phenyl, more especially 3,4-dichlorophenyl, 4-bromophenyl, 2-fluorophenyl, 4-fluorophenyl, 3-chlorophenyl, 4-chlorophenyl, especially C₁₋₆alkoxy phenyl, more especially 3-methoxyphenyl, 4-methoxyphenyl, 3,4-dimethoxyphenyl, especially cyanophenyl, more especially 2-cyanophenyl; toluyl, especially Het-substituted toluyl, more especially 3-(pyridin-2-yl)toluyl; naphthylene, especially naphthyl-2-ene; benzoyl, especially 2-benzoyl; benzo[1,3]dioxolyl, especially benzo[1,3]dioxol-5-yl; benzo[1,2,5]oxadiazolyl, especially benzo[1,2,5]oxadiazolyl; pyridinyl, especially pyridin-2-yl, pyridin-3-yl, especially 1-oxy-pyridinyl, more especially 1-oxy-pyridin-2-yl, 1-oxy-pyridin-3-yl; especially C₁₋₆alkylpyridinyl, more especially 3-methyl-pyridin-2-yl, 6-methyl-pyridin-2-yl, thiophenyl, especially thiophene-2-yl; thiazolyl, especially thiazol-2-yl; 1H-imidazolyl, especially 1H-imidazol-2-yl, 1H-imidazol-4-yl, more especially C₁₋₆alkyl substituted imidazolyl, even more especially 1-methyl-1H-imidazol-2-yl, 1-methyl-1H-imidazolyl; 1H-[1,2,4]triazolyl, especially 1H-[1,2,4]triazol-3-yl, more especially C₁₋₆alkyl substituted 1H-[1,2,4]triazolyl, even more especially 5-methyl-1H-[1,2,4]triazol-3-yl; or quinolinyl; and;
R′is H.

Even yet more preferred are compounds of Formula I wherein:

R₁ is 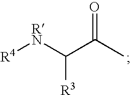
R₂ is C₃₋₆cycloalkyl-C₀₋₆alkyl or Ar—C₀₋₆alkyl;
R₃ is C₁₋₆alkyl;
R₄ is R₅C(O);
R₅ is Het-C₀₋₆alkyl;
R₉ is Het-C₀₋₆alkyl;
R′is H; and
R″ is H
Still yet more preferred are compounds of Formula I wherein: 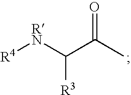
R₂ is C₃₋₆cycloalkyl-C₀₋₆alkyl particularly cyclohexyl, cyclohexylmethyl; or Ar—C₀₋₆alkyl, particularly benzyl;
R₃ is isobutyl;
R₄ is R₅C(O);
R₅ is hydrogen, 5-methoxybenzofuran-2-yl, benzo[b]thiophen-2-yl, 3-methyl-benzofuran-2-yl, thieno[3,2-b]thiophen-2-yl, benzofuran-2-yl, furo[3,2-b]pyridin-2-yl, 3-methyl-furo[3,2-b]pyridin-2-yl; preferably benzofuran-2-yl, furo[3,2-b]pyridin-2-yl, or 3-methyl-furo[3,2-b]pyridin-2-yl; most preferably benzofuran-2-yl.
R₉ is pyridin-2-yl or 1-oxy-pyridin-2-yl, preferably pyridin-2-yl.
R′is H; and
R″ is H.
   Synthetic Methods

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobrornic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li—⁺, Na⁺, K⁺, Ca⁺⁺, Mg⁺ and NH₄⁺ are specific examples of cations present in pharmaceutically acceptable salts. Halides, sulfates, phosphates, alkanoates (such as acetate and trifluoroacetate), benzoates, and sulfonates (such as mesylate) are examples of anions present in pharmaceutically acceptable salts.

This invention also provides a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated, as solutions or lyophilized powders for parenteral administration. Powders may be reconstituted by addition of a suitable diluent or other pharmaceutically acceptable carrier prior to use. The liquid formulation may be a buffered, isotonic, or aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

Utility of the Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds are useful for treating diseases in which cysteine proteases are implicated, including infections by pneumocystis carinji, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Parasites known to utilize cysteine proteases in their life cycle (and the diseases caused by these parasites) include Trypanosoma cruzi, Trypanosoma Brucei [trypanosomiasis (African sleeping sickness, Chagas disease)], Leishmania mexicana,—Leishmania pifanoi, Leishmania major (leishmaniasis), Schistosoma mansoni (schistosorniasis), Onchocerca volvulus [onchocerciasis (river blindness)] Brugia pahangi, Entamoeba histolytica, Giardia lambia, the helninths, Haemonchus contortus and Fasciola hepatica, as well as helminths of the genera Spirometra,—Trichinella, Necator and Ascaris, and protozoa of the genera Cryptosporidium,—Eimeria, Toxoplasma and Naegleria. The compounds of the present invention are suitable for treating diseases caused by these parasites which may be therapeutically modified by altering the activity of cysteine proteases. In particular, the present compounds are useful for treating malaria by inhibiting falcipain.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly cysteine proteases of the papain superfamily, yet more particularly cysteine proteases of the cathepsin family, which methods comprise administering to an animal, particularly a mammal, most particularly a human in need thereof a compound of the present invention. The present invention especially provides methods of treatment of diseases caused by pathological levels of cathepsin K, which methods comprise administering to an animal, particularly a mammal, most particularly a human in need thereof an inhibitor of cathepsin K, including a compound of the present invention. The present invention particularly provides methods for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

The present method provides treatment of diseases (in parentheses) caused by infection by Trypanosoma cruzi, Trypanosoma Brucei [trypanosomiasis (African sleeping sickness, Chagas disease)], Leishmania mexicana, Leishmania pifanoi,—Leishmania major (leishmaniasis), Schistosoma mansoni (schistosomiasis), Onchocerca volvulus [onchocerciasis (river blindness)] Brugia pahangi, Entamoeba histolytica,—Giardia lambia, the heiminths, Haemonchus contortus and Fasciola hepatica, as well as helminths of the genera Spirometra, Trichinella, Necator and Ascaris, and protozoa of the genera Cryptosporidium, Eimeria, Toxoplasma and Naegleria by inhibiting cysteine proteases of the papain superfamily by administering to a patient in need thereof, particularly an animal, more particularly a mammal, most particularly a human being, one or more of the above-listed compounds.

Most particularly, the present invention provides a method of treating malaria, caused by infection with Plasmodium falciparum, by the inhibition of falcipain by administering to a patient in need thereof, particularly an animal, more particularly a mammal, most particularly a human being, one or more of the above-listed compounds.

The present method may be practiced by administering the above-listed compounds alone or in combination, with each other, or with other therapeutically effective compounds.

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

Bioassay

The compounds of this invention may be tested in one of several biological assays to determine the concentration of compound which is required to have a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 uM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):
v=VₘA/[Kₐ(1+I/K_{i,app})+A] (1)
where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:
[AMC]=vₛₛ t+(v₀−vₛₛ)[1−exp (−k_{obs}t)]/k_{obs} (2)
where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[1]) describing the time-dependent inhibition. A complete discussion of this kinetic treatment has been fully described (Morrison et al., Adv. Enzymol. Relat. Areas Mol. Biol., 1988, 61, 201).
Human Osteoclast Resorption Assay

Aliquots of osteoclastoma-derived cell suspensions were removed from liquid nitrogen storage, warmed rapidly at 37° C. and washed ×1 in RPMI-1640 medium by centrifugation (1000 rpm, 5 min at 4° C.). The medium was aspirated and replaced with murine anti-HLA-DR antibody, diluted 1:3 in RPMI-1640 medium, and incubated for 30 min on ice The cell suspension was mixed frequently.

The cells were washed ×2 with cold RPMI-1640 by centrifugation (1000 rpm, 5 min at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 min on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated ×10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7 g/litre of sodium bicarbonate. 3 mL aliquots of the cell suspension (per treatment) were decanted into 15 mL centrifuge tubes. These cells were pelleted by centrifugation. To each tube 3 mL of the appropriate treatment was added (diluted to 50 uM in the EMEM medium). Also included were appropriate vehicle controls, a positive control (87MEM1 diluted to 100 ug/mL) and an isotype control (IgG2a diluted to 100 ug/mL). The tubes were incubate at 37° C. for 30 min.

Aliquots (0.5 mL) of the cells were seeded onto sterile dentine slices in a 48-well plate and incubated at 37° C. for 2 h. Each treatment was screened in quadruplicate. The slices were washed in six changes of warm PBS (10 mL/well in a 6-well plate) and then placed into fresh treatment or control and incubated at 37° C. for 48 h. The slices were then washed in phosphate buffered saline and fixed in 2% glutaraldehyde (in 0.2M sodium cacodylate) for 5 min., following which they were washed in water and incubated in buffer for 5 min at 37° C. The slices were then washed in cold water and incubated in cold acetate buffer/fast red garnet for 5 min at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API m, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

Methods of Preparation and Specific Examples

Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

The following Scheme I illustrates one process for preparing the compounds of this invention.

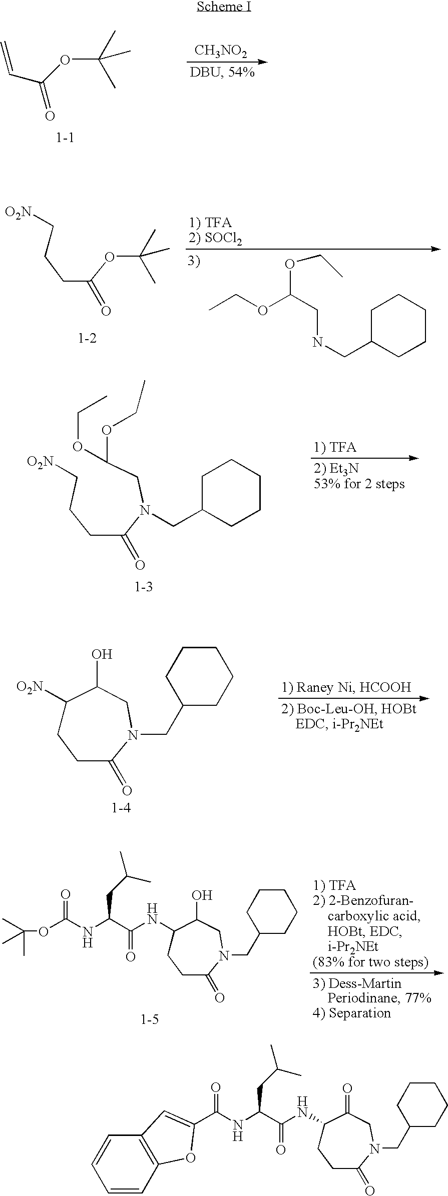

The acrylate starting material (1-1) is available commercially (Aldrich). Michael addition effects adding nitromethane to the tert-butyl acrylate (1-2). The acid chloride is then formed by first saponifying the ester and treating that product with thionyl chloride. It is treated with a substituted aminoacetaldehyde dimethyl acetal to give 1-3. The acetal is converted to the aldehyde and the crude aldehyde with an organic base effects the nitro-aldol ring closure to provide the 5-nitroazepane-2-one (1-4). Reduction of the nitro group, illustrated here by the use of Raney Ni, in the presence of an acid provides an amine which is then coupled with N-Boc-leucine in the presence of a coupling agent common to the art, such as EDC-HCl (1-5). Deprotection of the Boc group followed by acylation with an acylating agent such as benzofuran-2-carboxylic adic, HOBT, EDC and tertiary amine provides a 3-hydroxy-7-oxoazepan-4-yl intermediate which is then oxidized (Dess-Martin periodinane) to give the final product, after appropriate purification.

This set of steps can be used to make other compounds of formula 1, by simply varing the starting material or the penultimate ester-forming step. In addition the synthetic processes described in the PCT application having publication number WO 01-70232 published 27 Sep. 2001 can be used to make compounds of this invention as well. Those chemistries are incorporated herein by reference in full.

The following specific examples are provided to illustrate the invention further. They are representative examples, and are not intended to limit the invention. Reference is made to the claims for what is reserved to the inventors hereunder.

### EXAMPLES

### Example 1

### Preparation of Benzofuran-2-carboxylic acid [(S)-1-((S)-1-cyclohexylmethyl-3,7-dioxo-azepan-4-ylcarbamoyl)-3-methyl-butyl]-amide

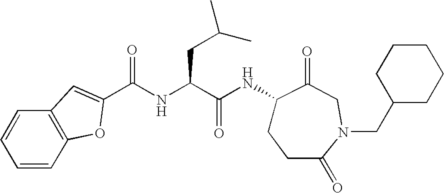

### 1a. 4-Nitro-butyric acid t-butyl ester

To a solution of tert-butyl acrylate (10 g, 78.0 mmol) in CH₃NO₂ was added DBU and stirred at ambient temperature. After 1.5 hr, the reaction mixture was quenched with sat'd NH₄Cl and extracted with diethyl ether (200 ml×2). The organic layer was washed with 2N HCl (100 ml), sat'd NaHCO₃ (100 ml), then dried over MgSO₄. After evaporation of solvent on rotovap, the residue was purified by vacuum distillation (bath 130-150° C., 5 mmHg) to give the tide compound (8.01 g, 54%); ¹H NMR (CDCl₃): δ 1.47 (s, 9H), 2.29 (m, 2H), 2.38 (t, J=6.8 Hz, 2H), 4.48 (t, J=6.7 Hz, 2H).

### 1b. 4-Nitrobutyric acid

To a solution of 4-nitro-butyric acid t-butyl ester (3.15 g, 16.7 mmol) in CH₂Cl₂ (10 ml) was added TFA (10 ml). After 1.0 hr at RT, the solvent and TFA were evaporated under reduced pressure to yield title compound. The resultant residue was subjected to the next reaction without further purification.

### 1c. 4-Nitro-butyryl chloride

4-Nitrobutyric acid (from step b) was cooled to 0° C. and treated with SOCl₂ (7.0 ml). The mixture was stirred at 0° C. for 10 min and then it was heated to 81° C. After 1 h, the mixture was concentrated under reduced pressure and azeotroped with benzene to yield the title compound as a solid residue used in the next reaction without further purification.

### 1d. N-Cyclohexylmethyl-N-(2,2-diethoxy-ethyl)-4-nitro-butyramide

4-Nitro-butyryl chloride (from step c) was dissolved in CH₂Cl₂ (40 ml) and cooled to 0° C. To this solution was added cyclohexylmethyl-aminoacetaldehyde dimethyl acetal (3.35 g, 16.7 mmol), followed by triethylamine (2.8 ml, 20 mmol). After 2 h at 0° C., the reaction was quenched with cold 1N HCl and extracted with CH₂Cl₂ (50 ml×2). The organic layer was washed with sat'd NaHCO₃ (60 ml), brine (60 ml), and then dried over MgSO₄. Purification on silica gel column yielded the title compound (4.1 g, 78% for three steps); ¹H NMR (CDCl₃): δ 0.90 (m, 2H), 1.20 (m, 4H), 1.50-1.85 (m, 5H), 2.35 (m, 2H), 2.50 (m, 2H), 2.60 (m, 1H), 3.20 (d, J=7.3 Hz, 2H), 3.26 (d, J=7.05 Hz, 1H), 3.37 (d, J=5.2 Hz, 1H), 3.40 (s, 6H), 4.54 (t, J=6.5, 2H); LCMS: 317 (MH⁺).

### 1e. N-Cyclohexylmethyl-4-nitro-N-(2-oxo-ethyl)-butyramide

To N-cyclohexylmethyl-N-(2,2-diethoxy-ethyl)-4-nitro-butyramide in CH₂Cl₂ (10 ml) was added trifluoroacetic acid (10 ml). The mixture was stirred at ambient temperature for 2 h. After the reaction mixture was concentrated in vacuo, saturated NaHCO₃ was added to the residue and then it was extracted with CH₂Cl₂ (50 ml ×3). The organic layer was washed with brine and then dried over MgSO₄. After concentration in-vacuo, the title compound obtained was used next step without purification; LCMS: 271 (MH⁺).

### 1f. N-Cyclohexylmethyl-6-hydroxy-5-nitro-azepan-2-one

To a solution of crude N-cyclohexylmethyl-4-nitro-N-(2-oxo-ethyl)-butyramide (1.9 g, 7.01 mmol) in THF (15 ml) was added triethylamine (15 ml) at ambient temperature and the mixture was stirred overnight. The mixture was then concentrated in-vacuo and purified on silica-gel column to give 1.01 g (53%) of title compound; ¹H NMR (CDCl₃): δ 1.00 (m, 2H), 1.10-1.30 (m, 3H), 1.50-1.80 (m, 6H), 2.1-2.2 (m, 1H), 2.45-2.70 (m, 3H), 3.10-3.20 (m, 1H), 3.30-3.45 (m, 2H), 3.55-3.65 (m, 1H), 4.15 (m, 1H), 4.45 (m, 1H); LCMS: 271 (MH⁺).

### 1 g. 5-Amino-N-Cyclohexylmethyl-6-hydroxy-5-nitro-azepan-2-one

To a solution of N-cyclohexylmethyl-6-hydroxy-5-nitro-azepan-2-one (0.40 g, 1.48 mmol) in CH₃OH (2 ml) was added Raney nickel (about 6 ml of settled solid) and the suspension was stirred for 5 min at room temperature. Formic acid (5 ml) was then added to the suspension and stirring was continued for 2.5 hr. The mixture was filtered through celite. The solid was washed with MeOH and the filtrate was concentrated in-vacuo. The residue was resuspended in water and basified to pH 10-11 followed by extraction with CHCl₃ (with 10% ethanol, 100 ml×4). The combined organic was dried over MgSO₄ and concentrated in-vacuo. The title compound obtained was used in next step without purification; LCMS: 241 (MH⁺).

### 1 h. [(S)-1-(1-Cyclohexylmethyl-3-hydroxy-7-oxo-azepan-4-ylcarbamoyl)₃-methyl-butyl]-carbamic acid tert-butyl ester

To a solution of 5-amino-N-cyclohexylmethyl-6-hydroxy-5-nitro-azepan-2-one (196 mg, 0.82 mmol) in CH₂Cl₂ (2.5 ml) was added BOC-(L)-leucine (208 mg, 0.90 mmol), 1-hydroxybenzotriazole (127 mg, 0.94 mmol), EDC-HCl (180.5 mg, 0.94 mmol) followed by triethylamine (0.28 ml, 2.0 mmol) at RT. After stirring overnight at RT, CH₂Cl₂ was removed by rotovap, diluted with ethylacetate, washed with water, brine, dried over MgSO₄, filtered, and concentrated by rotary evaporation. The residue was purified by silica gel column (0% to 2% MeOH/CH₂Cl₂) to yield the title compound (250 mg, 68%); ¹H NMR (CDCl₃): δ 0.97 (m, 8H), 1.20 (m, 4H), 1.46 (s, 9H), 1.55-1.85 (m, 10H), 2.40-2.75 (m, 2H); 3.10-3.70 (m, 4H), 3.80-4.15 (m, 2H), 4.75-4.95 (m, 1H); LCMS: 454 (MH⁺).

### 1i. Benzofuran-2-carboxylic acid [(S)-1-((3S,4S)-1-cyclohexylmethyl-3-hydroxy-7-oxo-azepan-4-ylcarbamoyl)-3-methyl-butyl]-amide

HCl (4M in dioxane, 2.76 ml, 11.0 mmol) was added to a solution of [(S)-1-(1-cyclohexylmethyl-3-hydroxy-7-oxo-azepan-4-ylcarbamoyl)-3-methyl-butyl]-carbamic acid tert-butyl ester (250 mg, 0.55 mmol) in MeOH (3 ml) and stirred for 1 h at RT. The mixture was then concentrated in-vacuo and azeotroped with toluene three times. The resultant solid was then dissolved in DMF followed by the addition of 2-benzofurancarboxylic acid (94 mg, 0.58 mmol), 1-hydroxybenzotriazole (82 mg, 0.61 mmol), EDC-HCl (117 mg, 0.61 mmol), and N,N-diisopropylethylamine (0.29 ml, 1.66 mmol). After stirring overnight at RT, the reaction mixture was quenched with cold 1N HCl, and extracted with ethylacetate. The combined organic layer was washed with sat'd NaHCO₃, brine, dried over MgSO₄, and concentrated in-vacuo. The residue was purified on silica gel column to give the title compound (227 mg, 83%); ¹H NMR (CDCl₃): δ 1.01 (m, 10H), 1.19 (m, 4H), 1.45-1.95 (m, 7H), 1.95-2.15 (m, 1H), 2.40-2.70 (m, 2H), 3.00-3.10 (m, 1H), 3.15-4.05 (m, 5H), 4.45-4.65 (m, 1H), 6.60 (m, 1H), 6.85-7.05 (m, 1H), 7.33 (m, 2H), 7.35-7.60 (m, 2H), 7.70 (m, 1H); LCMS: 498 (MH⁺).

### 1j. Benzofuran-2-carboxylic acid[(S)-1-((S)-1-((S)-1-cyclohexylmethyl-3,7-dioxo-azepan-4-ylcarbamoyl)-3-methyl-butyl]-amide

Dess-Martin periodinane (370 mg, 0.873 mmol) was added to a solution of benzofuran-2-carboxylic acid [(S)-1-((3S,4S)-1-cyclohexylmethyl-3-hydroxy-7-oxo-azepan-4-ylcarbamoyl)-3-methyl-butyl]-amide (217 mg, 0.44 mmol) and stirred at room temperature for 1.5 h. The reaction was then quenched with 10% aq. Na₂S₂O₃ and sat'd NaHCO₃. The aqueous layer was extracted with CH₂Cl₂ and the combined organic layers were washed with brine, dried over MgSO₄, and concentrated in-vacuo. The residue was purified by silica gel column to give the title compound (166 mg, 77%), which was further purified on a chiral column (S,S′-ULMO on Gilson HPLC) to separate the two diastereomers (first eluting, 56 mg, second eluting 43 mg); ¹H NMR (CDCl₃): δ 0.95 (m, 8H), 1.0-1.25 (m, 4H), 1.55-1.80 (m, 9H), 2.45-2.70 (m, 3H), 3.0-3.10 (m, 1H), 3.35-3.50 (m, 1H), 3.85-3.95 (m, 1H), 4.0-4.10 (m, 1H), 4.65 (m, 1H), 4.95 (m, 1H), 6.66 (d, 1H), 6.91 (d, 1H), 7.23 (m, 1H), 7.34-7.47 (m, 3H), 7.60 (d, J=7.8 Hz, 1H); LCMS: 496 (MH⁺).

### Example 2

### Preparation of Benzofuran-2-carboxylic acid [(S)-1-((S)-1-benzyl-3,7-dioxo-azepan-4-ylcarbamoyl)-3-methyl-butyl]-amide

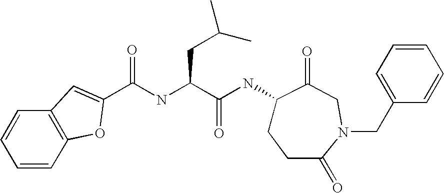

Following the procedure of Example 1 (a-d), except substituting benzyl-aminoacetaldehyde dimethyl acetal for cyclohexylmethyl-aminoacetaldehyde dimethyl acetal gave the title compound: ¹H NMR (CDCl₃, 400 MHz): δ 0.90 (m, 7H), 1.60-1.69 (m, 3H), 2.50-2.75 (m, 3H), 3.80-4.00 (m, 2H), 4.50-4.70 (m, 3H), 4.80-4.95 (m, 1H), 6.65 (m, 1H), 6.90 (m, 1H), 7.15-7.30 (m, 6H), 7.30-7.50 (m, 3H), 7.60 (d, J=7.8 Hz, M1); LCMS: 490 (MH⁺).

### Example 3

### Preparation of Benzofuran-2-carboxylic acid [(S)-1-((S)-1-cyclohexyl-3,7-dioxoazepan-4 -ylcarbamoyl)-3-methyl-butyl]-amide

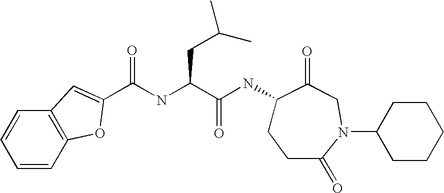

Following the procedure of Example 1 (a-d), except substituting cyclohexyl-aminoacetaldehyde dimethyl acetal for cyclohexylmethyl-aminoacetaldehyde dimethyl acetal gave the title compound: ¹H NMR (CDCl₃, 400 MHz): δ 0.92 (m, 7H), 1.0-1.80 (m, 13H), 2.3-2.70 (m, 3H), 3.65-3.90 (m, 2H), 4.30-4.50 (m, 1H), 4.50-4.70 (m, 1H), 4.80-5.00 (m, 1H), 6.60 (m, 1H), 6.90 (m, 1H), 7.20 (m, 1H), 7.30-7.50 (m, 3H), 7.60 (d, J=7.7 Hz, 1H); LCMS: 482 (MH⁺).

### Example 4

### Preparation of Benzofuran-2-carboxylic acid [(S)-1-((S)-1-methyl-3,7-dioxoazepan-4-ylcarbamoyl)-3-methyl-butyl]-amide

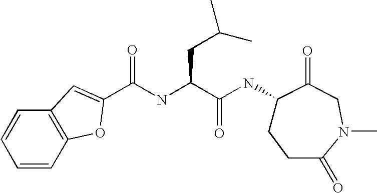

Following the procedure of Example 1 (a-d), except substituting methyl-aminoacetaldehyde dimethyl acetal for cyclohexylmethyl-aminoacetaldehyde dimethyl acetal gave the title compound. The diastereomers were not separated; ¹H NMR (CDCl₃, 400 MHz): δ 0.90 (m, 7H), 1.65 (m, 3H), 2.40-2.80 (m, 3H), 3.00 (s, 3H), 3.95-4.10 (m, 2H), 4.65 (m, 1H), 4.90 (m, 1H), 6.70-6.90 (m, 2H), 7.25 (m, 1H), 7.25-7.45 (m, 3H), 7.60 (d, J=7.8, 1H); LCMS: 414 (MH⁺).

1. a compound according to Formula I. 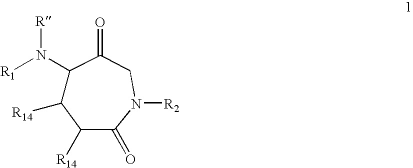
wherein:
R₁ is either formula A or B 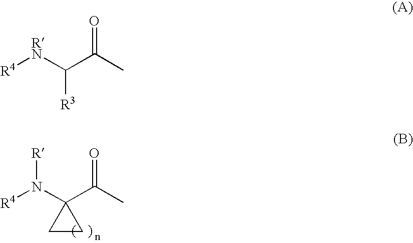
wherein in formula (B), n is an integer from 1 to 5;
R₂ is H, C₁₋₆alkyl, C₃₋₆cycloalkyl-C₀₋₆alkyl, Ar—C₀₋₆alkyl, Het-C₀₋₆alkyl, R₉C(O)—,
R₉C(S)—, R₉SO₂—, R₉OC(O)—, 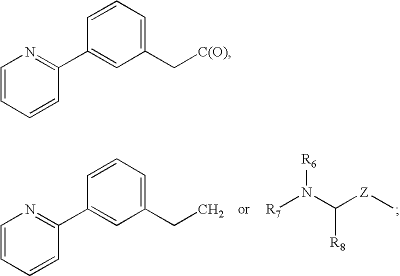
R₃ is H, C₁ allyl, C₃₋₆cycloalkyl-C₀₋₆alkyl, C₂₋₆alkenyl, C₂₋₆alkynyl, HetC₀₋₆alkyl, ArC₀₋₆alkyl, Ar—ArC₀₋₆, Ar-HetC₀₋₆alkyl, Het-ArC₀₋₆alkyl, or Het-HetC₀₋₆alkyl;
R₃ and R′ may be connected to form a pyrrolidine, piperidine or morpholine ring;
R₄ is C₁₋₆alkyl, C₃₋₆cycloalkyl-C₀₋₆alkyl, Ar—C₀₋₆alkyl, Het-C₀₋₆alkyl, R₅C(O), R₅—C(S)—, R₅SO₂—, R₅OC(O)—, R₅R₁₂NC(O)—, or R₅R₁₂NC(S);
R₅ is H, C₁₋₆alkyl, C₂₋₆alkenyl, C₂₋₆alkynyl, C₃₋₆cycloalkyl-C₀₋₆alkyl, C₂₋₆-alkanonyl, Ar—C₀₋₆alkyl, Het-C₀₋₆alkyl Ar—ArC₀₋₆alkyl, Ar-HetC₀₋₆alkyl, Het-ArC₀₋₆alkyl, or Het-HetC₀₋₆alkyl;
R₆ is H, C₁₋₆alkyl, Ar—C₀₋₆alkyl, or Het-C₀₋₆alkyl;
R₇ is H, C₁₋₆alkyl, C₃₋₆cycloalkyl-C₀₋₆alkyl, Ar—C₀₋₆alkyl, Het-C₀₋₆alkyl, R₁₀C(O)—, R₁₀C(S)—, R₁₀SO₂—, R₁₀OC(O)—, R₁₀R₁₃NC(O)—, or R₁₀R₁₃NC(S)—;
R₈ is H, C₁₋₆alkyl, C₂₋₆alkenyl, C₂₋₆alkynyl, Ar—C₀₋₆alkyl or Het-C₀₋₆alkyl;
R₉ is H, C₁₋₆alkyl, C₃₋₆cycloalkyl-C₀₋₆alkyl, C₂₋₆alkenyl, C₂₋₆alkynyl, Ar—C₀₋₆alkyl or Het-C₀₋₆allyl;
R₁₀ is C₁₋₆alkyl, C₃₋₆cycloalkyl-C₀₋₆alkyl, Ar—C₀₋₆alkyl or Het-C₀₋₆alkyl;
R₁₁ is H, C₁₋₆alkyl, Ar—C₀₋₆alkyl, C₃₋₆cycloalkyl-C₀₋₆alkyl, or Het-C₀₋₆alkyl;
R₁₂ is H, C₁-alkyl, Ar—C₀₋₆alkyl, or Het-C₀₋₆alkyl;
R₁₃ is H, C₁₋₄alkyl, Ar—C₀₋₆alkyl, or Het-C₀₋₆-alkyl;
each R₁₄ is independently H, C₁-alkyl, OC₁₋₄alkyl, SC₁₋₄alkyl, N(C₁₋₄alkyl)₂, —CH₂OC₁₋₄alkyl, CH₂SC₁₋₄alkyl, CH₂N(R₁₂)₂, Ar—C₀₋₆alkyl or Het-C₀₋₆alkyl;
R′ is H, C₁₋₆alkyl, Ar—C₀₋₆alkyl, or Het-C₀₋₆alkyl;
R″ is H, C₁-alkyl, Ar—C₀₋₆alkyl, or Het-C₀₋₆alkyl;
Z is C(O) or CH₂; or
a pharmaceutically acceptable salt, hydrate or solvate thereof.

2. A compound according to claim 1 wherein R₁ is

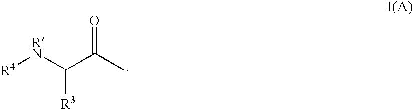

3. A compound according to claim 1 wherein R₂ is C₃₋₆cycloalkyl-C₀₋₆alkyl, or Ar—C₀₋₆alkyl.

4. A compound according to claim 2 wherein R₃ is C₁₋₆alkyl, C₃₋₆cycloalkyl-C₀₋₆alkyl, or ArC₀₋₆ alkyl.

5. A compound according to claim 2 wherein R₃ is H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluyl, naphthalen-2-ylmethyl, benzyloxymethyl, and hydroxymethyl.

6. A compound according to claim 2 wherein R₃ is toluyl, isobutyl or cyclohexylmethyl.

7. A compound according to claim 2 wherein R₃ is isobutyl.

8. A compound according to claim 1 wherein R₄ is RC(O), R₅C(S)—, R₁₄SO₂—.

9. A compound according to claim 8 wherein R₅ is C₁₋₆alkyl, C₂₋₆alkenyl, C₃₋₆cycloalkyl-C₀₋₆alkyl, C₂₋₆-alkanonyl, Ar—C₀₋₆alkyl, or Het-C₀₋₆alkyl.

10. A compound according to claim 9 wherein R₅ is:
methyl, halogenated methyl, C₁₋₆ alkoxy and aryloxy substituted methyl, heterocycle substituted methyl;
butyl, aryl substituted butyl;
isopentyl;
cyclohexyl;
butenyl, aryl substituted butenyl;
pentanonyl;
phenyl, phenyl substituted with one or more halogens, phenyl substituted with one or more C₁₋₆ alkoxy groups, phenyl substituted with one or more sulfonyl groups;
benzyl;
naphthylenyl;
benzo[1,3]dioxolyl;
furanyl, halogen substituted furanyl, aryl substituted furanyl;
tetrahydrofuranyl;
benzofuranyl, C₁₋₆alkoxy substituted benzofuranyl, halogen substituted benzofuranyl, C₁₋₆alkyl substituted benzofuranyl;
benzo[b]thiophenyl, C₁₋₆ alkoxy substituted benzo[b]thiophenyl;
quinolinyl;
quinoxalinyl;
1,8-naphthyridinyl;
indolyl, C₁₋₆alkyl substituted indolyl;
pyridinyl, C₁₋₆alkyl substituted pyridinyl, 1-oxy-pyridinyl;
furo[3,2-b]pyridinyl, C₁₋₆alkyl substituted furo[3,2-b]pyridinyl;
thiophenyl, C₁₋₆alkyl substituted thiophenyl, halogen substituted thiophenyl;
thieno[3,2-b]thiophenyl;
isoxazolyl, C₁₋₆alkyl substituted isoxazolyl; or
oxazolyl.

11. A compound according to claim 10 wherein R₅ is:
4-pentanonyl;
naphthylen-2-yl;
benzo[1,3]dioxol-5-yl,
tetrahydrofuran-2-yl
furan-2-yl;
benzofuran-2-yl;
benzo[b]thiophen-2-yl;
quinolin-2-yl, quinolin-3-yl, quinolin-4-yl, quinolin-6-yl, and quinolin-8-yl;
quinoxalin-2-yl;
1,8-naphthyridin-2-yl;
indol-3-yl, indol-5-yl;
pyridin-2-yl, pyridin-5-yl;
furo[3,2-b]pyridin-2-yl;
thiophen-3-yl;
thieno[3,2-b]thiophene-2-yl;
isoxazol-4-yl; or
oxazol-4-yl.

12. A compound according to claim 1 wherein formula 1 is 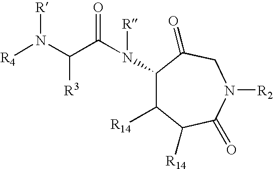
wherein each R₁₄ group is hydrogen.

13. A compound according to claim 12 which is:
benzofuran-2-carboxylic acid [(S)-1-((S)-1-cyclohexylmethyl-3,7-dioxo-azepan-4-ylcarbamoyl)-3-methyl-butyl]-amide;
benzofuran-2-carboxylic acid [(S)-1-((S)-1-benzyl-3,7-dioxo-azepan-4-ylcarbamoyl)₃-methyl-butyl]-amide;
benzofuran-2-carboxylic acid [(S)-1-((S)-1-cyclohexyl-3,7-dioxoazepan-4-ylcarbamoyl)-3-methyl-butyl]-amide; or
a pharmaceutically acceptable salt thereof.

14. A pharmaceutical preparation comprising a compound according to claim 1 and a pharmaceutically acceptable excipient.

15. A method for inhibiting a protease comprising administering to a patient in need thereof an effective amount of a compound according to claim 1.

16. A method according to claim 15 wherein said protease is a cysteine protease or a serine protease.

17. A method according to claim 15 wherein said protease is a cysteine protease.

18. A method according to claim 17 wherein said cysteine protease is cathepsin K.

19. A method according to claim 17 wherein the cysteine protease is falcipain.

20. A method of treating a disease characterized by bone loss comprising inhibiting said bone loss by administering to a patient in need thereof an effective amount of a compound according to claim 1.

21. A method according to claim 20 wherein said disease is osteoporosis.

22. A method according to claim 20 wherein said disease is periodontitis.

23. A method according to claim 20 wherein said disease is gingivitis.

24. A method of treating a disease characterized by excessive cartilage or matrix degradation comprising inhibiting said excessive cartilage or matrix degradation by administering to a patient in need thereof an effective amount of a compound according to claim 1.

25. A method according to claim 24 wherein said disease is osteoarthritis.

26. A method according to claim 24 wherein said disease is rheumatoid arthritis.

27. A method of treating a disease characterized by infection by a parasite selected from the group consisting of: Plasmodium falciparum, Trypaizosoma cruzi, Trypanosoma Brucei, Leishmatnia mexicana, Leishmania pifanoi, Leishmania nzajor, Schistosoma mansoni, Onchocerca volvulus, Brugia pahangi, Entamoeba histolytica, Giardia lamblia, the helminths Haemonchus contortus and Fasciola hepatica, the helminths of the genera Spirometra, Trichinella, Necator and Ascaris, and protozoa of the genera Cryptosporidiutm, Einieria, Toxoplasma and Naegleria, comprising inhibiting expression of a cysteine protease causing said disease by administering to a patient in need thereof an effective amount of a compound according to any one of claim 1.

28. A method according to claim 27 wherein said disease is selected from the group consisting of: malaria, trypanosomiasis (African sleeping sickness, Chagas disease), leishmaniasis, schistosomiasis, onchocerciasis (river blindness) and giardiasis.

29. A process for the synthesis of a compound according to claim 1 comprising the step of oxidizing a compound of formula II 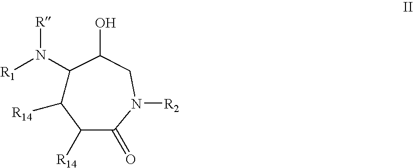
where the definitions of the depicted radicals R₁ etc are defined in claim 1, with an oxidizing agent to provide compounds of formula I as a mixture of diastereomers.

30. The process of claim 29 wherein the oxidizing agent is sulfur dioxide-pyridine complex or Dess-Martin periodinane.

31. The process of claim 29 further comprising the steps of separating the diasteromers by separating means.

32. The process of claim 31 wherein said separating means is high presssure liquid chromatography (HPLC).
